(19) United States
(12) Patent Application Publication
(10) Pub. No.: US 2019/0247618 A1
(43) Pub. Date: Aug. 15, 2019

(54) WIRELESS GUIDEWIRE

(71) Applicant: QUALCOMM Incorporated, San Diego, CA (US)

(72) Inventors: Stephen Jay SHELLHAMMER, Ramona, CA (US); Rashid Ahmed Akbar ATTAR, San Diego, CA (US); Adam Edward NEWHAM, Poway, CA (US); William Henry VON NOVAK, San Diego, CA (US); Osvaldo ALCALA, Chula Vista, CA (US); Jorge GARCIA, San Diego, CA (US); Daniel BUTTERFIELD, Encinitas, CA (US); Ravindra SHENOY, Dublin, CA (US)

(21) Appl. No.: 16/268,355

(22) Filed: Feb. 5, 2019

Related U.S. Application Data
(60) Provisional application No. 62/628,762, filed on Feb. 9, 2018.

(51) Int. Cl. A61M 25/01; H02J 50/23; H02J 50/30; H01L 25/065; A61M 25/00; G08C 17/02

(52) U.S. Cl. CPC A61M 25/0105; H02J 50/23; H02J 50/30; H03M 5/12; A61M 25/0012; G08C 17/02; H01L 25/0652

(57) ABSTRACT

A medical system is provided. The medical system includes a guidewire configured to guide a catheter to a target location within a body, the guidewire including a sensor configured to collect sensor data indicative of a location within the body, and an electrical conductor configured to conduct electrical signals representing the sensor data. The medical system further includes a wireless transmitter and a first antenna electrically coupled with the sensor via the electrical conductor and configured to: receive the electrical signals representing the sensor data; generate, from the electrical signals, first wireless signals representing the sensor data; and transmit, via the first antenna, first wireless signals.

### CROSS-REFERENCE TO RELATED APPLICATIONS

This application claims the benefit of U.S. Provisional Application No. 62/628,762, filed Feb. 9, 2018, entitled “Wireless Guidewire,” which is incorporated herein by reference in its entirety.

### BACKGROUND

A guidewire is a medical device that can be inserted into a blood vessel of a body to provide a mechanical guide for delivering a catheter into the body. A guidewire may be in the form of a tube and include material that is stiff enough to be pushed but flexible enough to follow the curve of the blood vessel. The distal end of the guidewire can be steered by an operator to navigate the blood vessels in the body en route to a target location. After the distal end of the guidewire reaches the target location, a catheter can slide down around the guidewire to reach the target location to support a medical application, such as delivering a therapy (e.g., balloon angioplasty, placement of stents, administration of drugs, etc.). Imprecision and error in the navigation of the guidewire can cause the distal end of the guidewire to reach an unintended location. As a result, the catheter can also be guided to the unintended location, and improper delivery of the therapy by the catheter may result.

### SUMMARY

According to some embodiments, a medical system is provided. The medical system comprises a guidewire configured to guide a catheter to a target location within a body, the guidewire including a sensor configured to collect sensor data indicative of a location within the body, and an electrical conductor configured to conduct electrical signals representing the sensor data. The medical system further comprises a wireless transmitter and a first antenna electrically coupled with the sensor via the electrical conductor and configured to: receive the electrical signals representing the sensor data; generate, from the electrical signals, first wireless signals representing the sensor data; and transmit, via the first antenna, first wireless signals.

In some aspects, the wireless transmitter and the first antenna are included in the guidewire.

In some aspects, the medical system further comprises a wireless receiver configured to receive second wireless signals.

In some aspects, the wireless receiver comprises a second antenna, an optical sensor, a photovoltaic cell, or any combination thereof. The second wireless signals are configured to provide wireless electric power transfer to the guidewire.

In some aspects, the wireless receiver is included in the guidewire.

In some aspects, the medical system further comprises a frequency synthesizer configured to: generate clock signals using the second wireless signals as a reference; and provide the clock signals to the wireless transmitter to control a timing of transmission of the first wireless signals.

In some aspects, the medical system further comprises a data converter configured to: convert analog signals generated by the sensor to digital sensor data; and provide the digital sensor data to the wireless transmitter for transmission via the first antenna. The frequency synthesizer is configured to provide the clock signals to the data converter to control a timing of operations at the data converter.

In some aspects, the medical system further comprises a data frame generator configured to: generate a data frame comprising a frame sync field, an address field, and a data field, the frame sync field including synchronization data, the address field including an identifier to identify the digital sensor data, and the data field including the digital sensor data; and provide the data frame to the wireless transmitter for transmission via the first antenna.

In some aspects, the data field includes the sensor data encoded based on Manchester coding scheme.

In some aspects, the frame sync field includes a maximal length sequence.

In some aspects, the identifier included in the address field is generated based on a physically unclonable function (PUF) and is encoded based on Manchester coding scheme.

In some aspects, the medical system further comprises a modulator configured to: modulate the first wireless signals based on data included in the data frame; and provide the modulated first wireless signals to the wireless transmitter for transmission. The first wireless signals are modulated according to an on-off keying scheme.

In some aspects, the medical system further comprises a wireless power module. The wireless power module includes a tuning module electrically coupled with the second antenna, the tuning module having a tunable impedance to adjust a quantity of power transferred from the second antenna to the wireless power module.

In some aspects, the wireless power module further comprises a protection module coupled with the tuning module and configured to detune or short the second antenna based on an output voltage of the second antenna.

In some aspects, the wireless power module further comprises: a rectifier coupled with the protection module and configured to generate a set of direct current (DC) pulses from an output of the tuning module; and a filter capacitor configured to generate a filtered DC voltage from the set of DC pulses.

In some aspects, the wireless power module further comprises a regulator configured to generate the electric power based on the DC voltage.

In some aspects, the wireless transmitter is included in an integrated circuit package comprising a stack of integrated circuit dies.

In some aspects, the integrated circuit package has a non-rectangular cross-section profile to fit into the guidewire.

In some aspects, the medical system further comprises a circuit board to provide electrical coupling between the integrated circuit package and the electrical conductor of the guidewire. The integrated circuit package is electrically coupled to the circuit board based on bond wires, through vias, or any combination thereof.

In some aspects, the first antenna comprises one of: a simple dipole antenna, a folded dipole antenna, or a quarter wave antenna.

In some aspects, the second wireless signals include radio frequency signals. In some aspects, the second wireless signals include optical signals.

In some aspects, the medical system further comprises a monitor device configured to: receive the first wireless signals; extract the sensor data from the first wireless signals; and output the sensor data.

In some aspects, the monitor device comprises two antennae configured to transmit, respectively, the first wireless signals and second wireless signals at different polarizations.

In some aspects, the guidewire comprises a first segment housing the electrical conductor and a second segment housing the wireless transmitter; wherein the first segment is made of metallic material, and wherein the second segment is made of non-metallic material.

According to some embodiments, a medical system is provided. The medical system comprises means for guiding a catheter to a target location within a body; means for collecting sensor data indicative of a location within the body; means for conducting electrical signals representing the sensor data; means for receiving the electrical signals representing the sensor data; means for generating, from the electrical signals, first wireless signals representing the sensor data; and means for transmitting the first wireless signals.

In some aspects, the medical system further comprises means for receiving second wireless signals; means for generating electric power from the second wireless signals; and means for providing the electric power to the means for collecting sensor data indicative of a location within the body.

In some aspects, the second wireless signals comprise optical signals.

In some aspects, the medical system further comprises: means for receiving the first wireless signals; means for extracting the sensor data from the first wireless signals; and means for outputting the sensor data.

In some embodiments, a method of fabricating an integrated circuit package to be inserted into a guidewire is provided. The method comprises: forming an integrated circuit substrate core having a first through via and a cut-out space; mounting the integrated circuit substrate core on a first carrier substrate; forming a dual die having a first pad on a first surface and a second pad on a second surface, the first surface being opposite to the second surface; placing the dual die in the cut-out space, the second surface facing the first carrier substrate; performing a polymer lamination process to fill space and to encapsulate the dual die in the cut-out space with a polymer lamination; drilling a second through via through the polymer lamination to reach the first pad; forming a first input/output pad between the first through via and the second through via; mounting the integrated circuit substrate core on a second carrier substrate facing the first input/output pad and the first surface of the dual die; removing the first carrier substrate to expose the second pad on the second surface of the dual die; forming a second input/output pad between the second pad and the second through via; and removing the second carrier substrate.

### BRIEF DESCRIPTION OF DRAWINGS

Non-limiting and non-exhaustive aspects are described with reference to the following figures, wherein like reference numerals refer to like parts throughout the various figures unless otherwise specified.

FIG. 1A and FIG. 1B illustrate examples of applications of a guidewire according to embodiments of the present disclosure.

FIG. 2A and FIG. 2B illustrate examples of a guidewire according to embodiments of the present disclosure.

FIG. 3 illustrates an example of a medical system comprising the example guidewires of FIG. 2A and FIG. 2B according to embodiments of the present disclosure.

FIG. 4 illustrates an example of components of the example guidewires of FIG. 2A and FIG. 2B according to embodiments of the present disclosure.

FIG. 5 is an example block diagram of an electronic component of the example guidewires of FIG. 2A and FIG. 2B according to embodiments of the present disclosure.

FIG. 6 illustrates an example of a data structure transmitted by the example guidewires of FIG. 2A and FIG. 2B according to embodiments of the present disclosure.

FIG. 7 is an example block diagram of another component of the example guidewires of FIG. 2A and FIG. 2B according to embodiments of the present disclosure.

FIG. 8A, FIG. 8B, FIG. 8C, FIG. 8D, and FIG. 8E illustrate examples of placement of the components within the guidewires of FIG. 2A and FIG. 2B according to embodiments of the present disclosure.

FIG. 9 illustrates an example of a method of fabricating electronic components of the guidewires of FIG. 2A and FIG. 2B according to embodiments of the present disclosure.

FIG. 10A, FIG. 10B, and FIG. 10C illustrate the components involved in the method of FIG. 9, according to embodiments of the present disclosure.

### DETAILED DESCRIPTION

Several illustrative embodiments will now be described with respect to the accompanying drawings, which form a part hereof. The ensuing description provides embodiment(s) only, and is not intended to limit the scope, applicability or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

It will be understood by a person of ordinary skill in the art that, although the embodiments provided herein are directed toward medical applications, the techniques described herein may be utilized in other applications involving digital communication. Additionally, it will be understood that the frequencies provided in each of the figures are provided as non-limiting examples, where alternative embodiments may utilize different frequencies. Similar numbers to specific embodiments provided in the figures may also be altered, depending on desired functionality. A person of ordinary skill in the art will recognize many variations.

A guidewire is a medical device that can be inserted into a blood vessel of a body to provide a mechanical guide for delivering a catheter into the body. An operator can apply a force to the guidewire to advance and/or to turn the guidewire to maneuver guidewire in the blood vessels. When the distal end of the guidewire reaches a target location, a catheter can slide down around the guidewire to reach the target location to support a medical application, such as delivering a therapy (e.g., balloon angioplasty, placement of stents, administration of drugs, etc.).

To facilitate accurate placement of the guidewire and the catheter inside the body, the operator can monitor the location of the distal end of the guidewire in real-time while navigating the guidewire in the blood vessels towards the target location. One way of determining the location can be based on sensing the distal end of the guidewire. A guidewire may include a sensor in or near the distal end. The sensor can generate sensor data indicative of the target location. For example, a guidewire may be used to reach a lesion region or a constriction region within a blood vessel for balloon angioplasty. The lesion region or constriction region may exhibit a reduction in blood flow and a change in blood pressure. To detect the lesion region or constriction region, the guidewire may include a pressure sensor to sense the blood pressure as the guidewire moves through the blood vessels. The blood pressure information can be transmitted back to an operator of the guidewire. The operator can maneuver the guidewire until the blood pressure information indicates that the target lesion region or construction region is reached. Compared with other techniques such as imaging the guidewire inside the body using fluoroscopic imaging, sensor-based navigation can provide more accurate location information of the guidewire and at a much higher rate, while the navigation and maneuvering operations are not limited by the visibility of the guidewire inside the body.

The guidewire can be part of a medical system that also includes a monitoring system. Sensor data can be provided by the guidewire to the monitoring system, which can generate an output to the operator based on the sensor data indicating that the distal end of the guidewire reaches the target location. In one configuration, a proximal end of the guidewire may be coupled with the monitor system via one or more electrical wires. The electrical wires can supply electric power to the electronic components housed within the guidewire (e.g., sensors, processing circuits, etc.), and to transmit the sensor data to the monitor system. Such arrangements, however, can pose numerous challenges to the efficient and accurate placement of the guidewire and the catheter. First, the electrical wires add considerable weight at the end of the guidewire and can constrain the precise maneuvering of the guidewire. Second, after the distal end of the guidewire reaches the target location, the monitoring system needs to be disconnected from the proximal end of the guidewire, to allow the catheter to slide through the guidewire to the target location. But the disconnection action can move the distal end of the guidewire away from the target destination, which leads to error in the placement of the catheter. Moreover, as the monitoring system is disconnected from the guidewire and does not receive the latest sensor data from the guidewire, the operator may be unaware of the movement of the distal end of the guidewire from the target location and cannot correct the placement error.

Embodiments of the present disclosure provide a wireless guidewire that may address some or all of these issues. The wireless guidewire may include a sensor, such as a pressure sensor, a position sensor, etc., to generate sensor data about an environment in which a distal end of the wireless guidewire is located. The wireless guidewire can transmit the sensor data wirelessly to a monitor. The wireless guidewire can also receive a wireless signal and generate electric power from the wireless signal, and supply the electric power to the sensor and to other circuitries housed within the wireless guidewire.

Various techniques are proposed for wireless transmission of the sensor data and for generation of electric power at the wireless guidewire. In some examples, the wireless guidewire may include built-in antennae to transmit the sensor data wirelessly to the monitoring system, and to receive a wireless signal for electric power generation. In some examples, the antennae can be external to the wireless guidewire and can be coupled with the wireless guidewire via, for example, electrical wires, inductive coupling, etc. The wireless guidewire also houses an integrated circuit package including analog front end and processing circuits to support the wireless transmission and reception of signals. The integrated circuit package can include stacked dies having unequal die sizes to fit into the wireless guidewire, which may have a circular or oval cross-section. The integrated circuit package may include bond wires or drill vias for electrical connection with other components (e.g., sensors, antennae, etc.) of the wireless guidewire.

A wireless guidewire according to embodiments of the present disclosure can be maneuvered as a standalone medical device, thus avoiding the aforementioned problems associated with attaching the guidewire with an electrical wire (or other devices). For example, as soon as a distal tip of the wireless guidewire reaches the pre-determined location, the operator can slide the catheter through the wireless guidewire while the distal tip remains at the pre-determined location. As a result, the placement of can be performed more efficiently and accurately.

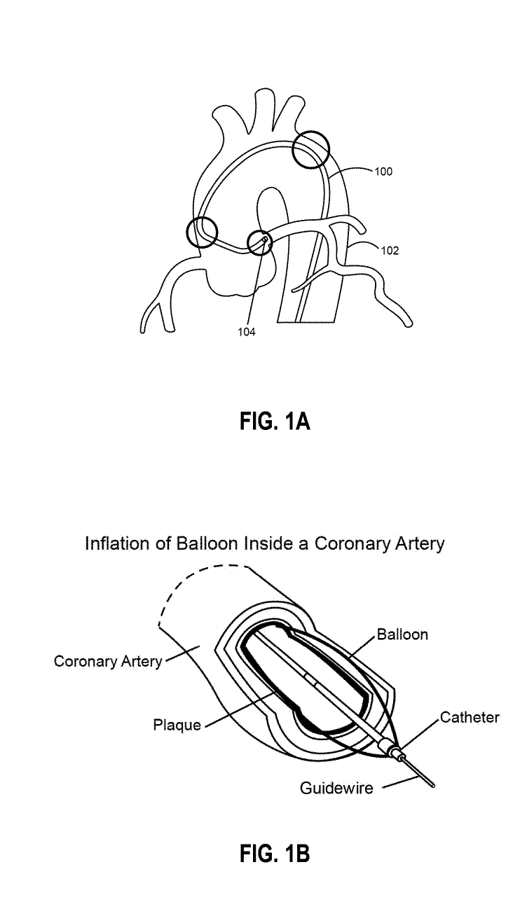

FIG. 1A illustrates an example of an application of a guidewire. In the example of FIG. 1A, a guidewire 100 can be controlled to navigate through a blood vessel 102 to reach a pre-determined location 104 within the body. In the example of FIG. 1A, guidewire 100 needs to be stiff enough to be pushed into blood vessel 102, yet at the same time guidewire 100 also needs to be flexible enough to follow the curve of blood vessel 102. Once a tip of the guidewire arrives at the destination, a soft catheter can slide down around the guidewire to be delivered to the destination to, for example, deliver a therapy such as balloon angioplasty, as shown in FIG. 1B.

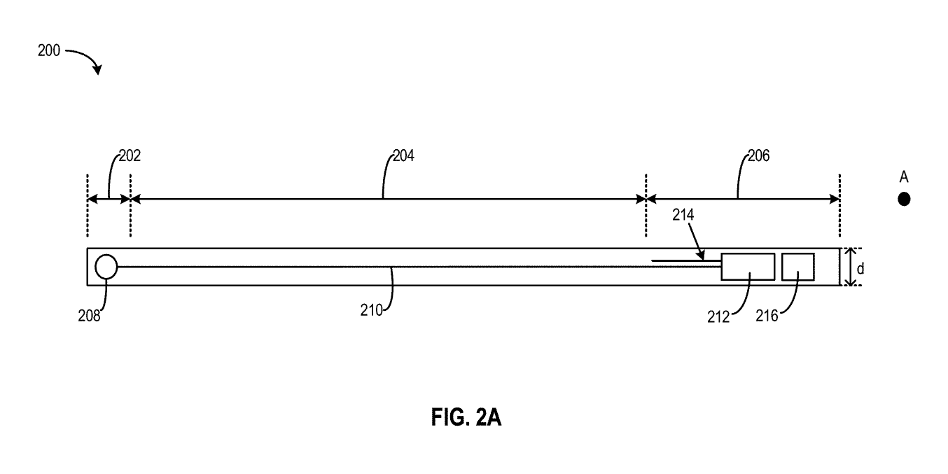

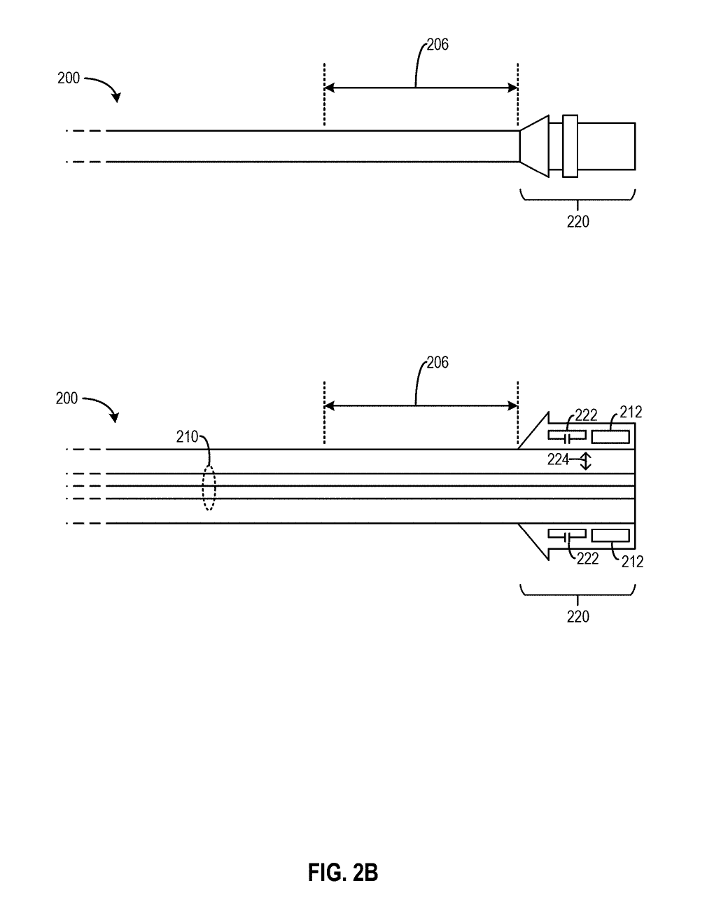

FIG. 2 illustrates an example of a wireless guidewire 200 according to embodiments of the present disclosure. In the example of FIG. 2, wireless guidewire 200 may include a segment 202, a segment 204, and a segment 206. Although FIG. 2 illustrates that wireless guidewire 200 includes three segments, it is understood that in other examples, wireless guidewire 200 may include more than three segments. Segment 202 may include a distal end (measured with reference to point A) to be inserted into a subject's blood vessel, and can house a sensor 208. Sensor 208 can include, for example, a pressure sensor, a position sensor, etc., that can provide sensor data to facilitate navigation through the blood vessels to reach a pre-determined destination within the subject's body. Segment 204 may be made with a flexible material that allows bending of the segment to follow the curve of a blood vessel. Segment 204 may house one or more electrical conductors 210. Segment 206 may house an electronic system 212, one or more antennae 214 and, optionally, a wireless power receiver 216. Segment 206 may be at a proximal end (labelled “A” in FIG. 2) of wireless guidewire 200.

Electronic system 212 may include circuits to support operations of wireless guidewire 200. As to be discussed in more detail below, electronic system 212 can obtain sensor signals from sensor 208 via electrical conductors 210, perform processing (e.g., analog-to-digital conversion, encoding and packetizing, etc.) and transmit the processed sensor signals wirelessly to a monitoring device (not shown in FIG. 2) using antennae 214. Antennae 214 can include, for example, one or more simple dipole antennae, one or more folded dipole antennae, one or more quarter wave antennae, or any combination thereof. In some examples, antennae 214 can be part of one or more electrical conductors 210. Radio frequency signals emitted by antennae 214 can propagate in the body via backscattering before reaching the monitor system. Moreover, in some examples, electronic system 212 can also generate location signals, such as ping signals, and broadcast the ping signals using antenna 214 to indicate the location of wireless guidewire 200. The ping signals allow a wireless power transmitter to focus wireless signals to wireless guidewire 200 to perform wireless electric power transfer. Electronic system 212 may include one or more integrated circuits housed within a package that fits within wireless guidewire 200, and may be electrically connected to electrical conductors 210 and antennae 214 via bond wires.

Wireless power receiver 216 can receive wireless signals, which can include radio frequency signals, optical signals, etc., from a wireless power transmitter as part of a wireless electric power transfer operation. The wireless power transmitter may include, for example, an infra-red illuminator, a visible light illuminator, a wireless charging station (e.g., a Powercast® transmitter), a beam-forming power transmitter (e.g., a Cota® system), etc. Wireless power receiver 216 can provide the wireless signals to electronic system 212, which can convert the received wireless signals to electric power, and supply the electric power to itself and via electrical conductors 210 to other components of guidewire 200, including sensor 208. Wireless power receiver 216 may include, for example, one or more antennae, optical sensors, matched photovoltaic (PV) cells, etc. In a case where wireless power receiver 216 includes antennae to receive radio frequency signals, electronic system 212 can also use the received radio frequency signals to generate reference clocks to control the timing of the sensor data processing operations.

In the example of FIG. 2A, at least segments 204 and 206 (and possibly segment 202) are configured to guide a catheter. Segments 202, 204, and 206 can be part of a single structure (e.g., a tube) with a uniform diameter d. However, it is understood that segments 202, 204, and 206 can be made of different materials, and may have different diameters. For example, as discussed above, segment 204 may be made with a flexible material. Segment 204 may also be coated with a layer of metallic material to form an electromagnetic (EM) shield to guard against EM interference. On the other hand, in some examples, segment 206 can be made with a rigid insulator material to provide structural support for electronic system 212. The insulator material may also allow radio frequency signals (transmitted by or to be received by antennae 214) to go through. In a case where a visible light signal is received for electric power transfer, the insulator material may also be transparent to allow visible light to go through. Moreover, the diameters of segments 202, 204, and 206 can also be different, provided that at least segments 204 and 206 are small enough to allow a catheter to slide over them.

Although FIG. 2A illustrates that electronic system 212, antennae 214, and power receiver 216 are housed within wireless guidewire 200, these components can be external to wireless guidewire 200. FIG. 2B illustrates examples of wireless guidewire 200 having electronic system 212, antennae 214, and power receiver 216 external the guidewire. As shown in FIG. 2B, wireless guidewire 200 can be coupled with a torque knob 220 to provide a handling surface for an operator to turn wireless guidewire 200 to navigate within the blood vessels. As shown in FIG. 2B, torque knob 220 may house tuned coils 222 which can operate as tuned repeaters to amplify transmitted signals (e.g., sensor data) and/or received signals (e.g., for wireless electric power transfer). Torque knob 220 may also house electronic system 212 and/or power receiver 216. Tuned coils 222 can be inductively coupled (represented by arrows 224) with electrical conductors 210 to receive the sensor data generated by sensor 208 and processed by electronic system 212, and transmit the sensor data wirelessly. Turned coils 222 can also receive wireless signals from a wireless power transmitter and provide the wireless signal to electronic system 212 to generate electric power. Electronic system 212 can transfer electric power to electrical conductors 210 via inductive coupling.

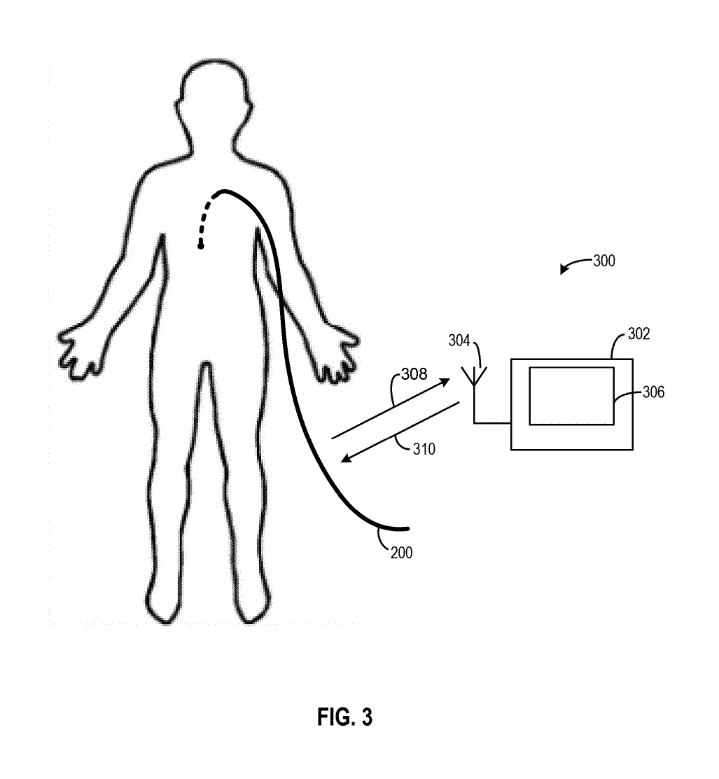

FIG. 3 illustrates an example of a medical system 300 according to embodiments of the present disclosure. In the example of FIG. 3, medical system 300 may include wireless guidewire 200 of FIG. 2A and FIG. 2B and a monitoring system 302. Monitoring system 302 may include one or more antennae 304 and an output interface 306. Monitoring system 302 can communicate wirelessly with wireless guidewire 200. For example, monitoring system 302 may include a receiver (not shown in FIG. 3) coupled with antennae 304 to receive sensor data 308 obtained by sensor 208. Monitoring system 302 can then output the sensor data (or information derived from the sensor data) using output interface 306 which may include, for example, a display screen as shown in FIG. 3, and/or indicator lights and audio speakers (not shown in FIG. 3), etc. Monitoring system 302 can also include a transmitter circuit (not shown in FIG. 3) coupled with antennae 304 to transmit wireless signals 310 to wireless guidewire 200. As to be discussed in more details below, wireless signals 310 may include, for example, control data to initiate a transmission of sensor data at wireless guidewire 200, and to set a frequency for the wireless transmission. Wireless signals 310 may also provide a clock signal reference for a frequency synthesizer at wireless guidewire 200 and can cause electric power generation at wireless guidewire 200.

In some examples, monitoring system 302 may transmit wireless signals 310 using different frequencies, and include one antenna (of antennae 304) for transmission of wireless signals 310 at a particular frequency. For example, monitoring system 302 may transmit wireless signals including control data (e.g., to initiate the wireless transmission of sensor data, to set the wireless transmission frequency, etc. at wireless guidewire 200) using a frequency of 915 MHz, which can be a frequency within the Industrial, Scientific, and Medical (ISM) radio frequency band. Monitoring system 302, or a standalone wireless power transmitter (not shown in FIG. 3) may also transmit the 915 MHz wireless signals with a high signal power to deliver electric power to wireless guidewire 200. On the other hand, monitoring system 302 may receive the sensor data from wireless guidewire 200 at a frequency of 403 MHz, which can be a frequency within the Medical Implant Communication Service (MICS) 402-405 MHz frequency band, to avoid interference by the much stronger 915 MHz wireless signals. In some examples, monitoring system 302 may include multiple sets of antennae, with each antenna configured to transmit wireless signals carrying the same information but with different polarizations. With such arrangements, the signal quality of wireless communications, and power to the wireless guidewire, between wireless guidewire 200 and monitoring system 302, can be maintained and are less susceptible to degradations caused by, for example, changes in the orientations of wireless guidewire 200 with respect to monitoring system 302. As a result, medical system 300 can become more robust.

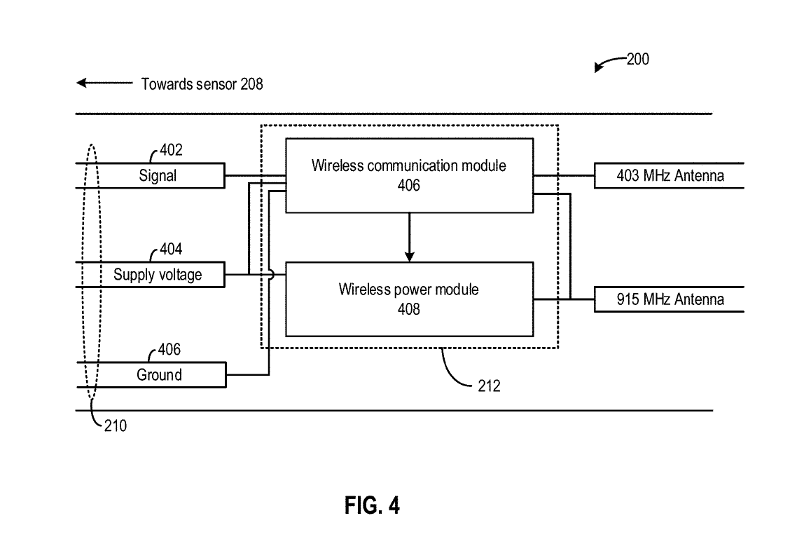

FIG. 4 illustrates an example of internal components of electrical conductors 210 and electronics system 212 of FIG. 2 according to embodiments of the present disclosure. In the example of FIG. 4, electrical conductors 210 include a signal conductor 402, a supply voltage conductor 404, and a ground conductor 405. Both signal conductor 402 and supply voltage conductor 404 may be electrically coupled with sensor 208 (not shown in FIG. 4). Signal conductor 402 may provide a path for sensor 208 to transmit sensor signals to electronic system 212, whereas supply voltage conductor 404 may be used to transmit electric power to sensor 208.

Electronics system 212 includes a wireless communication module 406 and a wireless power module 408. Wireless communication module 406 is coupled with signal conductor 402 and supply voltage conductor 404. Wireless communication module 406 is also coupled with a 403 MHz antenna and with a 915 MHz antenna (of antennae 214). Wireless communication module 406 may receive sensor signals from sensor 208 via signal conductor 402, process the signals to generate digital sensor data, and transmit the digital sensor data as 403 MHz wireless signals to monitoring system 302 using the 403 MHz antenna. Moreover, wireless communication module 406 may receive wireless signals at a frequency of 915 MHz using the 915 MHz antenna to extract the control data and to derive a reference clock signal from the 915 MHz wireless signals, and use the reference clock signal to synchronize the operations of various internal components of wireless communication module 406. On the other hand, wireless power module 408 may include circuits to extract and/or generate electric power from the 915 MHz wireless signals, and to transmit the electric power to wireless communication module 406 and sensor 208 via supply voltage conductor 404.

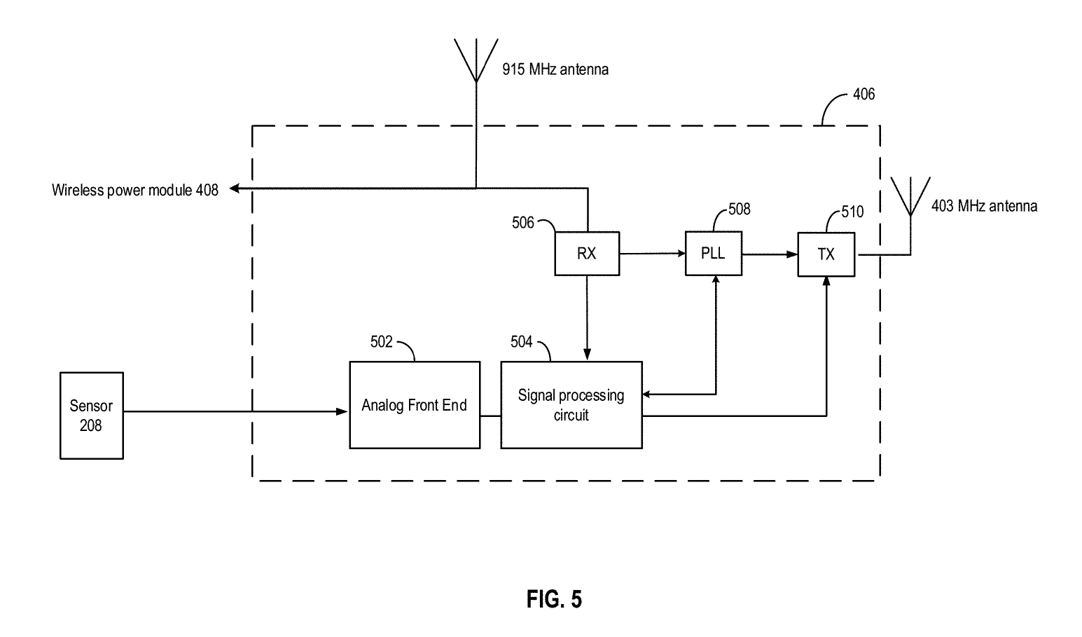

FIG. 5 illustrates an example of internal components of wireless communication module 406 according to embodiments of the present disclosure. In the example of FIG. 5, wireless communication module 406 may include an analog front end (AFE) 502, a signal processing circuit 504, a receiver (RX) 506, a frequency synthesizer (e.g., phase lock loop (PLL)) 508, and a transmitter (TX) 510.

In some examples, AFE 502 may include circuitries configured to receive sensor signals transmitted by sensor 208 and to perform analog signal conditioning operations on the receive signals. The signal conditioning operations may include, for example, amplification, filtering, etc. Signal processing circuit 504 may generate a digital representation of the sensor signals processed by AFE 502. For example, signal processing circuit 504 may include an analog-to-digital converter (ADC) to sample and quantize the sensor signals into a series of digital values. Signal processing circuit 504 may also perform additional processing such as generating data frames including the digital values and other information, and transmit the data frames to transmitter 510. Transmitter 510 can modulate a wireless carrier signal based on the data frames, and transmit the modulated wireless carrier signal using the 403 MHz antenna to monitoring system 302.

In some examples, receiver 506 can receive wireless signals from the 915 MHz antenna, and forward the wireless signals to PLL 508 as a reference signal, from which PLL 508 can generate a clock signal which may have the same or different frequency as the reference signal. PLL 508 can then forward the clock signal to signal processing circuit 504 and to transmitter 510 to control the timing of their operations.

For example, PLL 508 may set a sampling rate of the ADC at signal processing circuit 504. PLL 508 may also set a wireless carrier frequency at transmitter 510. Moreover, receiver 506 may also include a demodulator to extract the control data included in the wireless signals, and transmit the control data to signal processing circuit 504 to control its operation. For example, the control data may include an instruction to initiate signal processing circuit 504 to start transmitting data frames to transmitter 510 for transmission. The control data may also include an instruction to set a frequency of transmission of the data frames. Signal processing circuit 504 may forward the frequency of transmission information to PLL 508 to set the wireless carrier frequency for transmitter 510 to, for example, 403 MHz.

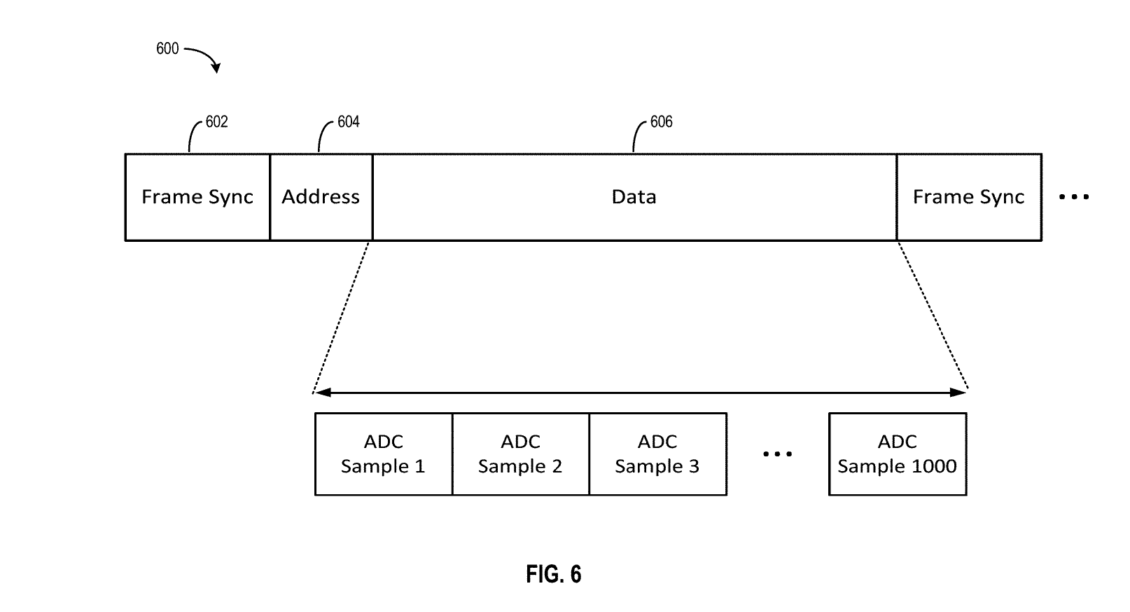

As discussed above, wireless communication module 406 may transmit the digitized sensor data in the form of data frames to monitoring system 302. FIG. 6 illustrates an example data frame sequence 600 that can be transmitted by wireless communication module 406 according to embodiments of the present disclosure. In the example of FIG. 6, data frame sequence 600 may include a sequence of digital data to be transmitted by transmitter 510. The sequence of digital data may be divided into a frame sync segment 602, an address segment 604, and a data segment 606. In the example of FIG. 6, data frame sequence 600 may include a repeating data frame sequence comprising the same frame sync segment 602 and address segment 604 but different data segment 606 (if, for example, the sensor data generated by sensor 208 changes with time).

Frame sync segment 602 can include a sequence of data that enables a receiving device (e.g., monitoring system 302) to detect the start of a frame, and to determine which of the received digital data correspond to subsequent segments (e.g., address segment 604 and data segment 606). In some examples, frame sync segment 602 may include a 32-bit Maximal Length sequence (MLS).

Address segment 604 may include an identifier that enables monitoring system 302 to identify the source of the received sensor data. For example, in a case where multiple wireless guidewires 200 are in use at the same time, monitoring system 302 may pick up wireless signals carrying sensor data from multiple wireless guidewires 200 within a certain range. Based on the identifier in address segment 604, monitoring system 302 can output the sensor data from the right wireless guidewire 200 while discarding the sensor data from other wireless guidewires 200. In some examples, the identifier included in address segment 604 can be a 24-bit random address generated by a physically unclonable function (PUF). In some examples, the identifier can be a static value associated with the guidewire. In some examples, the identifier can also be dynamic and a new identifier can be generated each time guidewire 200 is powered up. Guidewire 200 can then transmit the identifier to monitoring system 302 as part of a pairing process. In some examples, the identifier included in address segment 604 can be Manchester encoded to eliminate long strings of zeros and ones to facilitate receiver synchronization (e.g., by providing more signal transitions to determine the optimum timing to sample the eye of the wireless signals).

Data segment 606 may include multiple digital samples of sensor data generated by signal processing circuit 504. In the example of FIG. 6, data segment 606 may include up to 1000 digital samples of the sensor data. In some examples, the sensor data included in data segment 606 can also be Manchester encoded to eliminate long strings of zeros and ones to facilitate receiver synchronization.

As discussed above, transmitter 510 can transmit a data frame sequence by modulating a wireless carrier of a frequency (e.g., 403 MHz) set by, for example, PLL 508. There are various ways of performing modulation. In one example, an amplitude modulation scheme such as on-off keying (OOK) can be used where the transmission of the wireless carrier can be enabled or disabled to represent, respectively, a digital “one” or a digital “zero.” The advantage of amplitude modulation is that the transmission and reception (and subsequent extraction) of the digital data is less susceptible to phase noise, which enables the use of an ultra-low power frequency synthesizer/PLL 508. A non-coherent receiver can be employed monitoring system 302 to extract the digital data based on the detection (and non-detection) of the wireless carrier transmitted by wireless guidewire 200.

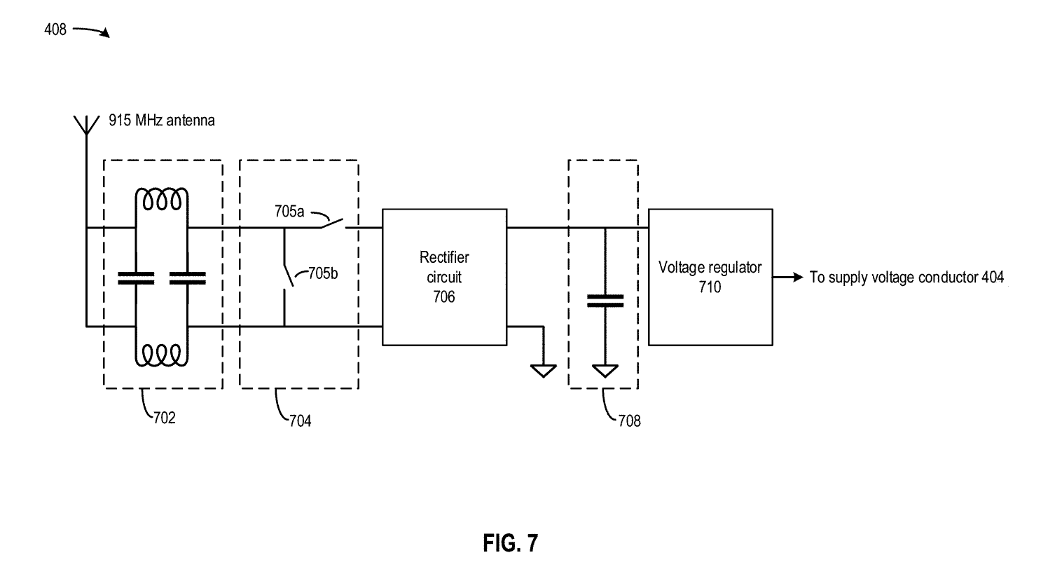

FIG. 7 illustrates an example of internal components of wireless power module 408 according to embodiments of the present disclosure. As discussed above, wireless power module 408 can extract and generate electric power from the 915 MHz wireless signals transmitted by monitoring system 302. In the example of FIG. 7, wireless power module 408 may include a tuning circuit 702, a protection circuit 704, a rectifier circuit 706, a filter capacitor 708, and a voltage regulator 710.

In some examples, tuning circuit 702 may include a set of inductors and variable capacitors to configure an input impedance of wireless power module 408. The input impedance may be configured to be, for example, 1000 ohm, to rematch the impedance of the 915 MHz antenna (which is typically 50 Ohm). Although the impedance change reduces the quantity of power transfer from the antenna to wireless power module 408, the impedance change can produce a relatively large voltage at the input of rectifier circuit 706, which can facilitate rectification by rectifier circuit 706 and subsequent regulation by voltage regulator 710. Although FIG. 7 illustrates that tuning circuit 702 includes two capacitors and two inductors, it is understood that tuning circuit 702 may include more or fewer inductors and capacitors than as shown in FIG. 7. Tuning circuit 702 may also include fewer (or none) of some of the components shown in FIG. 7.

Protection circuit 704 may include switches 705a and 705b which can detune or short the 915 MHz antenna when the antenna voltage becomes too high, which may cause damage (e.g., excessive voltage stress) to the rest of wireless power module 408. For example, the switches can be controlled by a voltage comparator (not shown in FIG. 7) which automatically closes switch 705b and/or opens switch 705a when the antenna voltage exceeds a pre-determined threshold. In some examples, Protection circuit 704 may also be used for signaling by, for example, changing the reflected impedance between the antenna and rectifier circuit 706 based on a data pattern. The changes in the reflected impedance can cause changes in the reflected wireless signals at the 915 MHz antenna and can lead to harmonic modulation. The harmonic modulation can be detected by nearby receiver devices, which can extract the data pattern based on the detected harmonic modulation.

Rectifier circuit 706 may include circuits to convert the antenna signal, which is an alternating current (AC) signal, into a direct current (DC) signal. The rectified signal may include a set of DC pulses. Rectifier circuit 706 may include a synchronous rectifier. The synchronous rectifier includes field effect transistors (FET) that can be turned on/off by the antenna signal. The rectified signal can be filtered by filter capacitor 708 to generate a smooth DC signal. The smooth DC signal can be fed as a voltage source to voltage regulator 710, which can generate a power supply voltage to be provided to sensor 208 and wireless communication module 406.

In some examples, voltage regulator 710 can be a switch-mode converter (e.g., a boost/buck regulator), a linear voltage regulator (e.g., a low-dropout (LDO) regulator), etc. Multiple voltage regulators 710 can be included to generate different supply voltages. For example, a 3.3V supply voltage can be supplied to sensor 508, whereas a 1.1V supply voltage can be supplied to other components of electronic system 212.

In FIG. 7, the inductor of tuning circuit 702 can include bond wires of a package that houses electronic systems 212, whereas the capacitors of tuning circuit 702, as well as capacitor 708, can include a metal-insulator-metal-insulator-metal (MIMIM) capacitor, a multi-layer ceramic capacitor (MLCC), a low profile silicon capacitor (LPSC), etc., which can be integrated within the package.

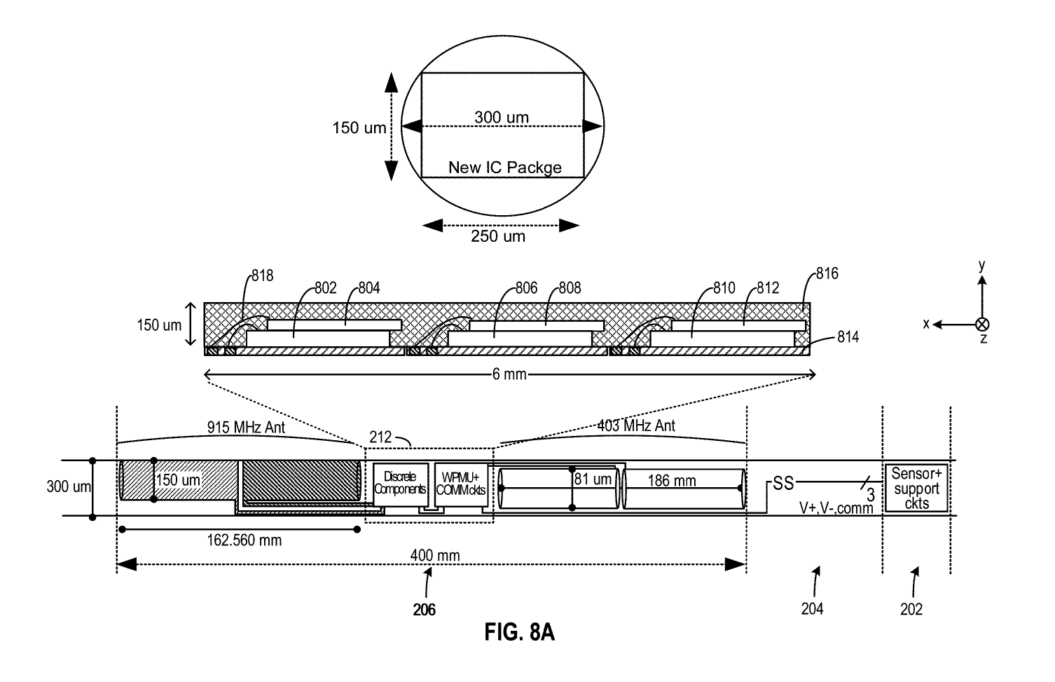

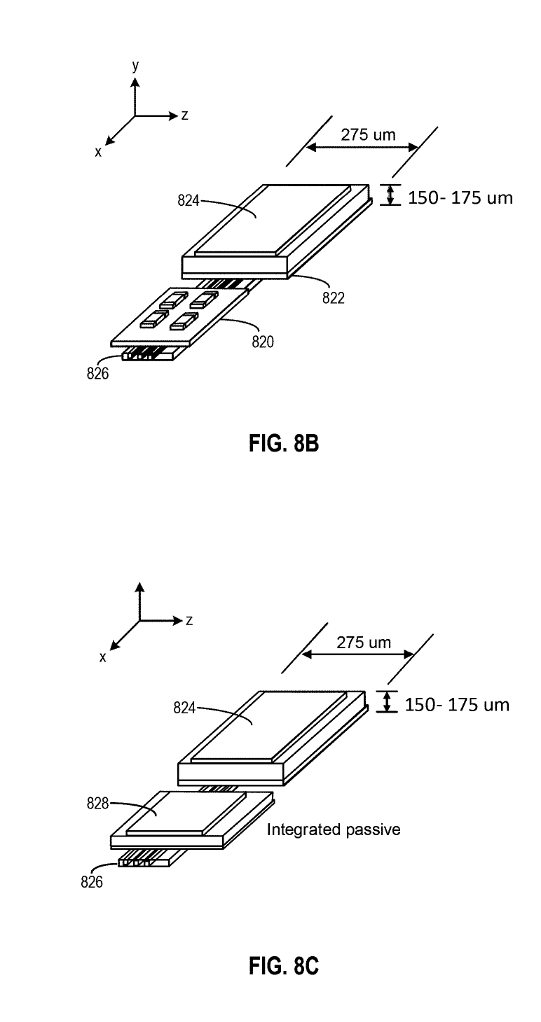

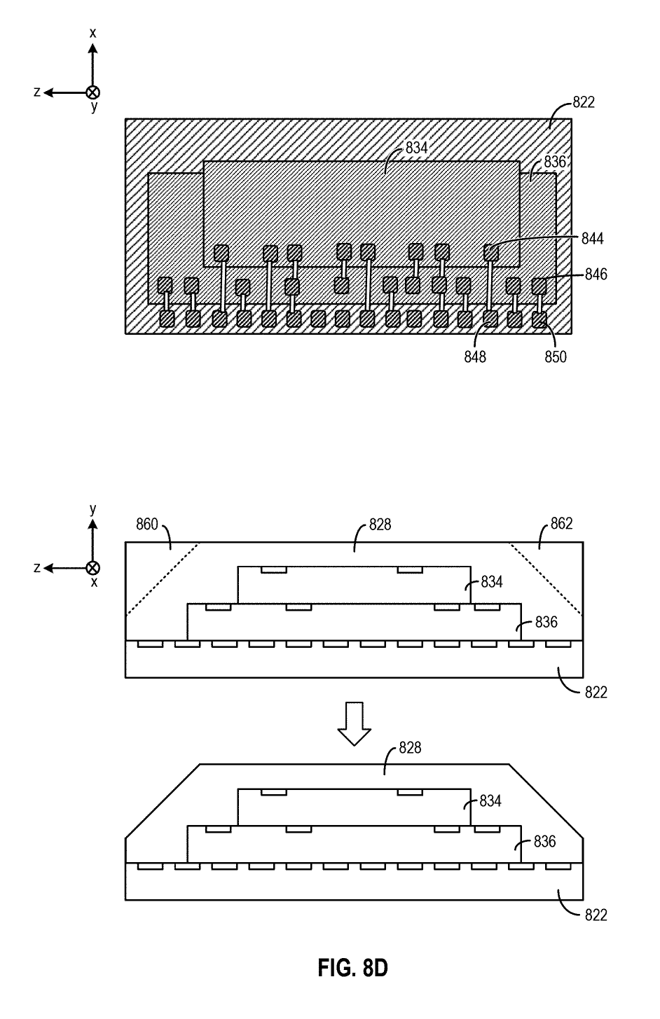

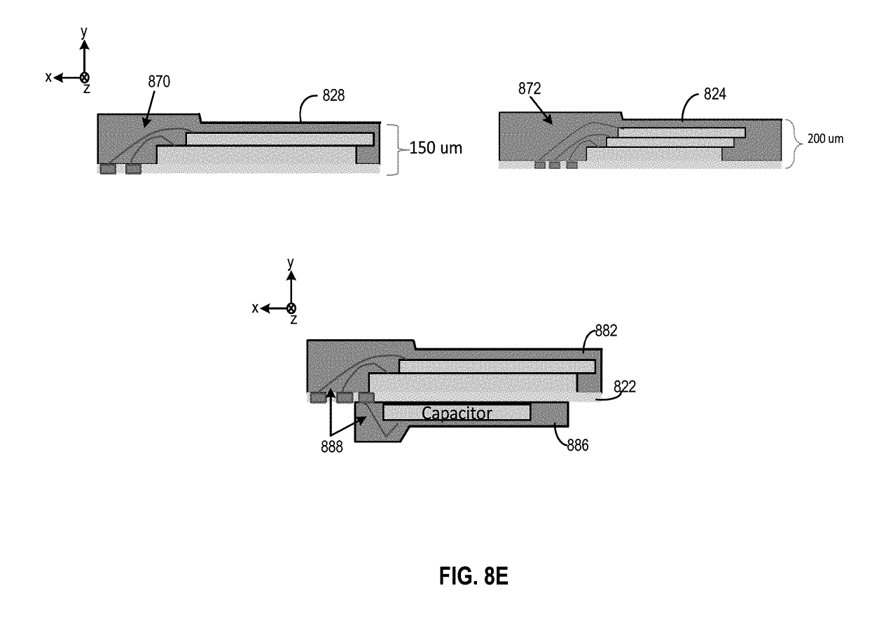

FIG. 8A illustrates examples of placement of internal components of wireless guidewire 200 according to embodiments of the present disclosure. In the example of FIG. 8, wireless guidewire 200 may have a diameter of 300 um (micro-meters) and can accommodate one or more integrated circuit (IC) packages with a height of 150 um and a width of 250 um. The integrated circuit package may include multiple integrated circuit dies to implement, for example, electronics system 212. In some examples, electronic systems 212 may include six integrated circuit dies 802, 804, 806, 808, 810 and 812 attached on a circuit board 814. The integrated circuit dies can be stacked. In the example of FIG. 8A, integrated circuit dies 802 and 804 can form a first stack, integrated circuit dies 806 and 808 can form a second stack, whereas integrated circuit dies 810 and 812 can form a third stack. Each stack can have a height between 150 um to 175 um. The first stack, second stack, and third stack can be lined up to form a row along the x-direction as shown in FIG. 8A. The row of stacks can be housed within an integrated circuit package 816. Each die can be coupled with circuit board 814 via one or more bond wires (e.g., bond wire 818). Integrated circuit package 816 may span a length of 6 mm (millimeters) and can be housed within segment 206 of wireless guidewire 200. Segment 206 also houses one or more 915 MHz antennae and 403 MHz antennae, as shown in the example of FIG. 8A.

FIG. 8B and FIG. 8C illustrates other examples of placement of the internal components of wireless guidewire 200 according to embodiments of the present disclosure. In the example of FIG. 8B, electronics system 212 can be formed on two circuit boards 820 and 822. Circuit board 820 may include discrete electronic components (e.g., capacitors, indictors, switches, other integrated circuits, etc.) for wireless power module 408. Circuit board 822 may be soldered to an integrated circuit package 824 including one or more integrated circuit dies for wireless communication module 406. Both circuit boards can stack on top of (and be electrically coupled to) a set of electrical conductors 826 including, for example, signal conductor 402, supply voltage conductor 404, and ground. In the example of FIG. 8C, the discrete components of wireless power module 408 can be replaced by integrated components, and electronics system 212 may include an integrated circuit package 828 for wireless power module 408 in addition to integrated circuit package 824 for wireless communication module 406.

FIGS. 8D and 8E illustrate examples of integrated circuit package 828 and the connections between the integrated circuit dies within the package and the circuit board (e.g., circuit board 822). The top diagram of FIG. 8D shows a top view of circuit board 822 and integrated circuit dies 834 and 836, whereas the bottom diagram shows a front view (e.g., in front of x-axis) of package 828 including circuit board 822 and integrated circuit dies 834 and 836. As shown in the top diagram of FIG. 8D, the integrated circuit dies 834 and 836 can form a stack along a vertical direction (e.g., along the y-axis). Each of integrated circuit dies 834 and 836, as well as circuit board 822, include conductive pads. For example, integrated circuit die 834 includes pad 844, and integrated circuit die 836 includes pad 846, whereas circuit board 822 includes pads 848 and 850. Each of integrated circuit dies 834 and 836 can be electrically connected to circuit board 822 (and to other electronic components) via bond wires formed between the pads, such as bond wire 852 between pads 844 and 848 and bond wire 854 between pads 846 and 850. Moreover, as shown in the bottom diagram of FIG. 8D, as integrated circuit dies 834 and 846 have different sizes, package 828 can be made of a trapezoid shape (e.g., by trimming away portions 860 and 862) to fit within the oval/circular cross section of wireless guidewire 200.

FIG. 8E illustrates some other examples of integrated circuit package 828. FIG. 8E illustrates side views (e.g., in front of the x-axis) of integrated circuit packages 824 and 828. For example, as shown in the top diagram of FIG. 8E, the integrated circuit packages can also have a non-rectangular profile along the x-direction (e.g., having different heights at different points along the x-direction). The non-rectangular profile can be adopted to provide additional space for the bond wires, such as bond wires 870 and 872. In another example, as shown in the bottom diagram of FIG. 8E, one integrated circuit package 882 (e.g., for wireless communication module 406) can be positioned on a top side of circuit board 822, whereas another integrated circuit package 886 (e.g., for wireless power module 408 including integrated capacitors) can be positioned on a bottom side of circuit board 822. Both packages can have non-rectangular profiles along the x-direction to accommodate bond wires 888.

In the examples of FIG. 8A to FIG. 8E, the integrated circuit packages include bond wires to provide electrical connections between dies and the circuit board. In some other examples, the electrical connections can be provided by through vias. Compared with bond wires, through vias take less volume of space and can shrink the footprint of the integrated circuit package, which makes it easier to fit the integrated circuit package into wireless guidewire 200.

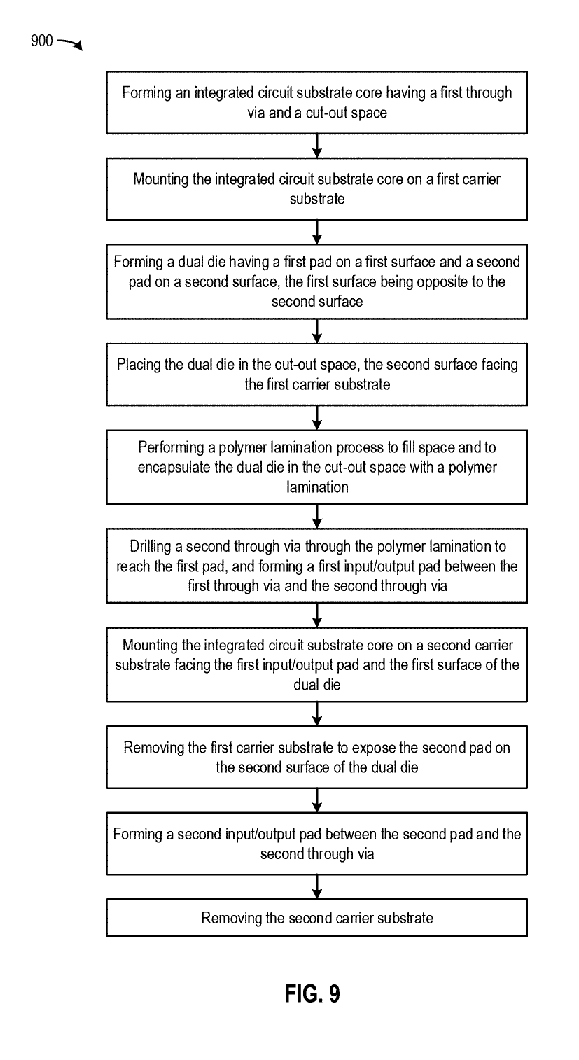

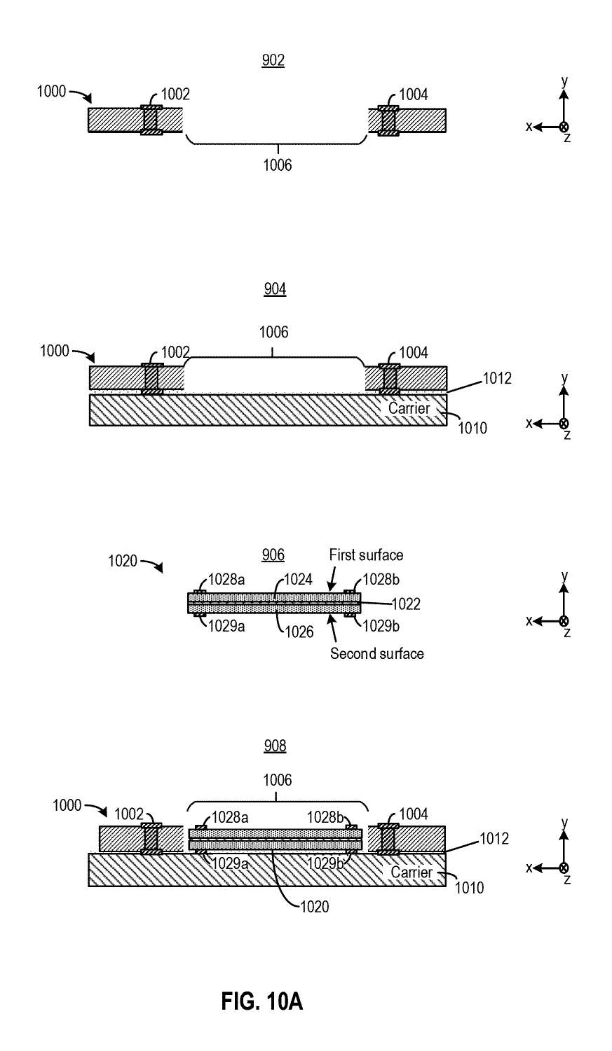

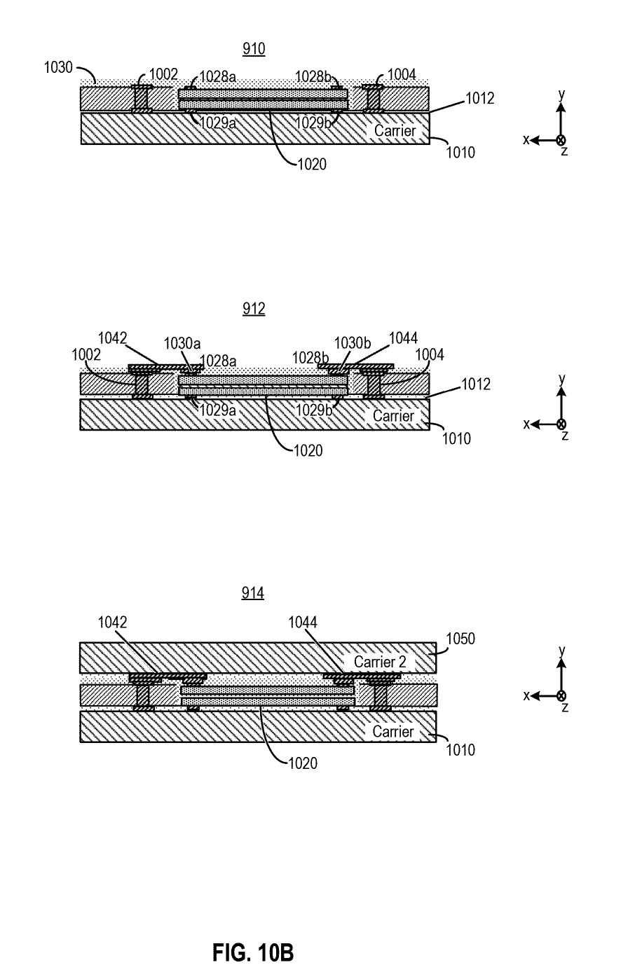

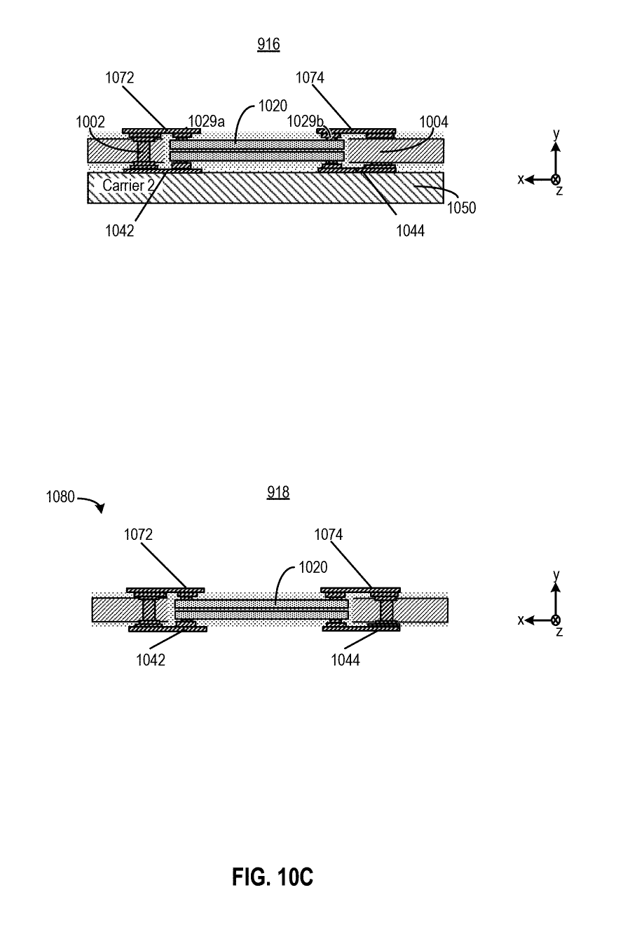

FIG. 9 illustrates a flowchart 900 of a method of fabricating an integrated circuit package having stacked dies, whereas FIG. 10A and FIG. 10B illustrate the components involved in the fabrication. Flowchart 900 can be used to fabricate an integrated circuit package which includes through vias to provide electrical connections between dies.

Referring to FIG. 9 and FIG. 10A, flowchart 900 starts with step 902, in which an integrated circuit substrate core 1000 having through a first through via (e.g., vias 1002 and 1004) and a cut-out space 1006 is formed. Integrated circuit substrate core 1000 can serve as a connection between the integrated circuit dies and a circuit board and can include a conductive network of traces and vias (e.g., via 1002).

In step 904, integrated circuit substrate core 1000 can be mounted on a first carrier substrate 1010. The mounting can be based on forming an adhesive layer 1012 between integrated circuit substrate core 1000 and first carrier substrate 1010.

In step 906, a dual die 1020 is formed. Dual die 1020 can be formed by having two wafers thinned and mounted back to back with an adhesive film 1022, followed by a singulation process to split the wafers into individual die pairs including dies 1024 and 1026. Each die can have a thickness of around 30-50 micrometers (um). Dual die 1020 includes a first surface having pads 1028a and 1028b and a second surface having pads 1029a and 1029b.

In step 908, dual die 1020 can then be placed in cut-out 1006. The second surface of dual die 1020 can face first carrier substrate 1010.

Referring to FIG. 9 and FIG. 10B, in step 910, a polymer lamination process can be performed to fill space and to encapsulate dual die 1020 with polymer lamination 1030.

In step 912, a second through via (e.g., vias 1030a and 1030b) can be drilled through polymer lamination 1030 to reach pads 1028a and 1028b of dual die 1020. In some examples, polymer lamination can include photo-imageable polymer, and the drilling of the through via lithography. In addition, a metal layer can be deposited on integrated circuit substrate core 1000 to form a redistribution layer (RDL) to form input/output pads 1042 and 1044 on a first surface of dual die 1020. As shown in FIG. 10B, input/output pad 1042 and via 1030a can provide an electrical connection between via 1002 and pad 1028a, whereas input/output pad 1044 and via 1030b can provide an electrical connection between via 1004 and pad 1028b.

In step 914, integrated circuit substrate core 1000 can be mounted on a second carrier substrate 1050 facing input/output pads 1042 and 1044 and the first surface of dual die 1020 having pads 1028a and 1028b. The attachment can be by forming a second adhesive film between second carrier 1050 and input/output pads 1042 and 1044.

Referring to FIG. 10C, in step 916, first carrier substrate 1010 can be removed (e.g., by weakening adhesive film 1012) to expose vias 1002 and 1004 as well as pads 1029a and 1029b on the second surface of dual die 1020. A RDL layer can be deposited to form input/output pads 1072 and 1074 on a second surface of dual die 1020.

In step 918, input/output pad 1072 can form an electrical connection between pad 1029a and via 1002, whereas input/output pad 1074 can form an electrical connection between pad 1029b and via 1004. In some examples, an assembly, including second carrier substrate 1050, integrated circuit substrate core 1000 including dual die 1020, and first carrier substrate 1010 can be flipped, and then first carrier substrate 1010 can be removed.

In step 920, second carrier substrate 1050 can then be removed from input/output pads 1042 and 1044, and an integrated circuit package 1080 including dual die 1020 is formed.

As shown in FIG. 10C, through vias can provide electrical connections to each die of dual die 1020 but can occupy much smaller space than bond wires, which can shrink the footprint of the integrated circuit package and make it easier to fit the integrated circuit package into wireless guidewire 200.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in certain implementations” or other like phrases in various places throughout this specification are not necessarily all referring to the same feature, example, and/or limitation. Furthermore, the particular features, structures, or characteristics may be combined in one or more examples and/or features.

Some portions of the detailed description included herein are presented in terms of algorithms or symbolic representations of operations on binary digital signals stored within a memory of a specific apparatus or special purpose computing device or platform. In the context of this particular specification, the term specific apparatus or the like includes a general-purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

In the preceding detailed description, numerous specific details have been set forth to provide a thorough understanding of claimed subject matter. However, it will be understood by those skilled in the art that claimed subject matter may be practiced without these specific details. In other instances, methods and apparatuses that would be known by one of ordinary skill have not been described in detail so as not to obscure claimed subject matter.

The terms, “and”, “or”, and “and/or” as used herein may include a variety of meanings that also are expected to depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein may be used to describe any feature, structure, or characteristic in the singular or may be used to describe a plurality or some other combination of features, structures or characteristics. Though, it should be noted that this is merely an illustrative example and claimed subject matter is not limited to this example.

While there has been illustrated and described what are presently considered to be example features, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter may also include all aspects falling within the scope of appended claims, and equivalents thereof.

For an implementation involving firmware and/or software, the methodologies may be implemented with modules (e.g., procedures, functions, and so on) that perform the functions described herein. Any machine-readable medium tangibly embodying instructions may be used in implementing the methodologies described herein. For example, software codes may be stored in a memory and executed by a processor unit. Memory may be implemented within the processor unit or external to the processor unit. As used herein the term “memory” refers to any type of long term, short term, volatile, nonvolatile, or other memory and is not to be limited to any particular type of memory or number of memories, or type of media upon which memory is stored.

In addition to storage on computer-readable storage medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims. That is, the communication apparatus includes transmission media with signals indicative of information to perform disclosed functions. At a first time, the transmission media included in the communication apparatus may include a first portion of the information to perform the disclosed functions, while at a second time the transmission media included in the communication apparatus may include a second portion of the information to perform the disclosed functions.

What is claimed is:

1. A medical system comprising:
a guidewire configured to guide a catheter to a target location within a body, the guidewire including a sensor configured to collect sensor data indicative of a location within the body, and an electrical conductor configured to conduct electrical signals representing the sensor data; and
a wireless transmitter and a first antenna electrically coupled with the sensor via the electrical conductor and configured to:
receive the electrical signals representing the sensor data;
generate, from the electrical signals, first wireless signals representing the sensor data; and
transmit, via the first antenna, first wireless signals.

2. The medical system of claim 1, wherein the wireless transmitter and the first antenna are included in the guidewire.

3. The medical system of claim 1, further comprising a wireless receive configured to receive second wireless signals.

4. The medical system of claim 3, wherein the wireless receiver comprises a second antenna, an optical sensor, a photovoltaic cell, or any combination thereof; and
wherein the second wireless signals are configured to provide wireless electric power transfer to the guidewire.

5. The medical system of claim 3, wherein the wireless receiver is included in the guidewire.

6. The medical system of claim 3, further comprising a frequency synthesizer configured to:
generate clock signals using the second wireless signals as a reference; and
provide the clock signals to the wireless transmitter to control a timing of transmission of the first wireless signals.

7. The medical system of claim 6, further comprising a data converter configured to:
convert analog signals generated by the sensor to digital sensor data; and
provide the digital sensor data to the wireless transmitter for transmission via the first antenna,
wherein the frequency synthesizer is configured to provide the clock signals to the data converter to control a timing of operations at the data converter.

8. The medical system of claim 7, further comprising a data frame generator configured to:
generate a data frame comprising a frame sync field, an address field, and a data field, the frame sync field including synchronization data, the address field including an identifier to identify the digital sensor data, and the data field including the digital sensor data; and
provide the data frame to the wireless transmitter for transmission via the first antenna.

9. The medical system of claim 8, wherein the data field includes the sensor data encoded based on Manchester coding scheme.

10. The medical system of claim 8, wherein the frame sync field includes a maximal length sequence.

11. The medical system of claim 8, wherein the identifier included in the address field is generated based on a physically unclonable function (PUF) and is encoded based on Manchester coding scheme.

12. The medical system of claim 8, further comprising a modulator configured to:
modulate the first wireless signals based on data included in the data frame; and
provide the modulated first wireless signals to the wireless transmitter for transmission,
wherein the first wireless signals are modulated according to an on-off keying scheme.

13. The medical system of claim 4, further comprising a wireless power module;
wherein the wireless power module includes a tuning module electrically coupled with the second antenna, the tuning module having a tunable impedance to adjust a quantity of power transferred from the second antenna to the wireless power module.

14. The medical system of claim 13, wherein the wireless power module further comprises a protection module coupled with the tuning module and configured to detune or short the second antenna based on an output voltage of the second antenna.

15. The medical system of claim 14, wherein the wireless power module further comprises:
a rectifier coupled with the protection module and configured to generate a set of direct current (DC) pulses from an output of the tuning module; and
a filter capacitor configured to generate a filtered DC voltage from the set of DC pulses.

16. The medical system of claim 15, wherein the wireless power module further comprises a regulator configured to generate the electric power based on the DC voltage.

17. The medical system of claim 1, wherein the wireless transmitter is included in an integrated circuit package comprising a stack of integrated circuit dies.

18. The medical system of claim 17, wherein the integrated circuit package has a non-rectangular cross-section profile to fit into the guidewire.

19. The medical system of claim 18, further comprising a circuit board to provide electrical coupling between the integrated circuit package and the electrical conductor of the guidewire,
wherein the integrated circuit package is electrically coupled to the circuit board based on bond wires, through vias, or any combination thereof.

20. The medical system of claim 1, wherein the first antenna comprises one of: a simple dipole antenna, a folded dipole antenna, or a quarter wave antenna.

21. The medical system of claim 3, wherein the second wireless signals include radio frequency signals.

22. The medical system of claim 3, wherein the second wireless signals include optical signals.

23. The medical system of claim 1, further comprising a monitor device configured to:
receive the first wireless signals;
extract the sensor data from the first wireless signals; and
output the sensor data.

24. The medical system of claim 23, wherein the monitor device comprises two antennae configured to transmit, respectively, the first wireless signals and second wireless signals at different polarizations.

25. The medical system of claim 1, wherein the guidewire comprises a first segment housing the electrical conductor and a second segment housing the wireless transmitter;
wherein the first segment is made of metallic material, and wherein the second segment is made of non-metallic material.

26. A medical system comprising:
means for guiding a catheter to a target location within a body;
means for collecting sensor data indicative of a location within the body;
means for conducting electrical signals representing the sensor data;
means for receiving the electrical signals representing the sensor data;
means for generating, from the electrical signals, first wireless signals representing the sensor data; and
means for transmitting the first wireless signals.

27. The medical system of claim 26, further comprising:
means for receiving second wireless signals;
means for generating electric power from the second wireless signals; and
means for providing the electric power to the means for collecting sensor data indicative of a location within the body.

28. The medical system of claim 27, wherein the second wireless signals include optical signals.

29. The medical system of claim 26, further comprising:
means for receiving the first wireless signals;
means for extracting the sensor data from the first wireless signals; and
means for outputting the sensor data.

30. A method of fabricating an integrated circuit package to be inserted into a guidewire, the method comprising:
forming an integrated circuit substrate core having a first through via and a cut-out space;
mounting the integrated circuit substrate core on a first carrier substrate;
forming a dual die having a first pad on a first surface and a second pad on a second surface, the first surface being opposite to the second surface;
placing the dual die in the cut-out space, the second surface facing the first carrier substrate;
performing a polymer lamination process to fill space and to encapsulate the dual die in the cut-out space with a polymer lamination;
drilling a second through via through the polymer lamination to reach the first pad;
forming a first input/output pad between the first through via and the second through via;
mounting the integrated circuit substrate core on a second carrier substrate facing the first input/output pad and the first surface of the dual die;
removing the first carrier substrate to expose the second pad on the second surface of the dual die;
forming a second input/output pad between the second pad and the second through via; and
removing the second carrier substrate.
